(19) United States
(12) Patent Application Publication
(10) Pub. No.: US 2024/0423943 A1
(43) Pub. Date: Dec. 26, 2024

(54) COMPOSITION FOR LIVING BODY

(71) Applicant: FUJIFILM Corporation, Tokyo (JP)

(72) Inventors: Haruki TOMIKAWA, Kanagawa (JP); Toshihide YOSHITANI, Kanagawa (JP)

(73) Assignee: FUJIFILM Corporation, Tokyo (JP)

(21) Appl. No.: 18/827,826

(22) Filed: Sep. 8, 2024

Related U.S. Application Data
(63) Continuation of application No. PCT/JP2023/008506, filed on Mar. 7, 2023.

(30) Foreign Application Priority Data
Mar. 28, 2022 (JP) 2022-051113

(51) Int. Cl. A61K 31/231; A61K 9/06; A61K 31/047; A61K 31/685

(52) U.S. Cl. CPC A61K 31/231; A61K 9/06; A61K 31/047; A61K 31/685

(57) ABSTRACT

In the present invention, there is provided a composition for a living body, which has excellent spreadability on an object, has a high film formation rate in a case of being brought into contact with water to form a film, increases the moisture retention of an object adjacent to the formed film, and has excellent strength of the formed film. The present invention relates to a composition for a living body, containing a neutral acyl lipid, a phospholipid, and an alcohol having 4 or less carbon atoms or a polyalkylene oxide, in which a content of a neutral diacyl lipid in the neutral acyl lipid is more than 0% by mass and 50% by mass or less, and a mass ratio of a content of the neutral acyl lipid to a content of the phospholipid is 54/46 to 30/70.

### CROSS-REFERENCE TO RELATED APPLICATIONS

This application is a Continuation of PCT International Application No. PCT/JP2023/008506 filed on Mar. 7, 2023, which claims priority under 35 U.S.C. § 119 (a) to Japanese Patent Application No. 2022-051113 filed on Mar. 28, 2022. The above applications are hereby expressly incorporated by reference, in their entirety, into the present application.

### BACKGROUND OF THE INVENTION

### 1. Field of the Invention

The present invention relates to a composition for a living body.

### 2. Description of the Related Art

In the related art, there is a demand for suppressing pain caused by inflammation or the like in a mucous membrane or a skin.

For example, in a cancer patient, cancer treatment affects the oral mucosa, which easily causes stomatitis. In anti-cancer drug treatment, in radiation therapy for head and neck cancer (cancer in the range from the head to the neck) in a case where a drug that easily causes stomatitis is administered, when radiation is directly applied to the mucous membrane of the mouth, stomatitis is inevitable. The pain of stomatitis is so strong that it is difficult to eat a meal by mouth.

As a symptomatic treatment for stomatitis, a patch (for example, Aphthaseal® 25 μg, manufactured by Taisho Pharmaceutical Co., Ltd., active ingredient: triamcinolone acetonide) that is directly attached to the affected part, an ointment (for example, Dexaltin oral ointment for oral cavity, manufactured by Nippon Kayaku Co., Ltd., active ingredient: dexamethasone) that is applied to the affected part, and a spray agent (for example, Salcoat® capsule for oral spray 50 μg, manufactured by TEIJIN PHARMA LIMITED, active ingredient: beclomethasone propionate ester) that is sprayed on the affected part are mentioned.

However, when eating a meal by mouth, the patch attached to the affected part may be peeled off, or the ointment or the spray agent applied to the affected part may be lost, and thus the pain of stomatitis cannot be suppressed.

On the other hand, as for the sustainability of the action of the composition to be locally applied, a preformulation in the form of a low viscosity mixture (molecular solution or the like) consisting of an amphiphilic component and at least one physiologically active substance optionally contained, in which in a case where the preformulation is exposed to an aqueous fluid such as body fluid, at least one phase transition is caused to form a bioadhesive matrix, is known.

For example, JP5144277B describes a formulation containing a low viscosity mixture of a) at least one neutral diacyl lipid containing at least 50% glyceryl dioleate, b) at least one phospholipid consisting of at least 50% phosphatidylcholine, and c) 2% to 30% by weight at least one biocompatible organic solvent containing ethanol, in which a weight ratio of the component a) to the component b) is 85:15 to 30:70, at least one bioadhesive liquid crystal phase structure can be formed by contact with an aqueous fluid and/or a body surface, and a viscosity is 0.1 to 5,000 mPas at 20° C.

### SUMMARY OF THE INVENTION

On the other hand, a composition for a living body to be applied to a living body is required to have various characteristics.

Specifically, in a case where the composition for a living body is applied to an object and is brought into contact with water in the atmosphere or the like to form a film, it is required that a formation rate of the film is high. In a case where the liquid crystal film (film exhibiting a liquid crystal phase) is formed by contact of the composition for a living body with water, a formation rate of the film corresponds to a rate at which the liquid crystal phase is formed.

In addition, it is also required that the composition for a living body has excellent spreadability in a case of applying the composition for a living body to an object. Furthermore, it is also required that the formed film can increase the moisture retention of an object adjacent to the formed film and the strength of the formed film itself is excellent.

As a result of studying the characteristics of the preformulation of the composition described in JP5144277B, the present inventors have found that all of the above-described specific characteristics cannot be simultaneously satisfied and further improvement is required.

An object of the present invention is to provide a composition for a living body, which has excellent spreadability on an object, has a high film formation rate in a case of being brought into contact with water to form a film, increases the moisture retention of an object adjacent to the formed film, and has excellent strength of the formed film.

As a result of intensive examination on the above-described problem, the present inventors found that the above-described problem can be solved by the following configurations.

[1] A composition for a living body, comprising a neutral acyl lipid, a phospholipid, and an alcohol having 4 or less carbon atoms or a polyalkylene oxide, in which a content of a neutral diacyl lipid in the neutral acyl lipid is more than 0% by mass and 50% by mass or less, and a mass ratio of a content of the neutral acyl lipid to a content of the phospholipid is 54/46 to 30/70.

[2] The composition for a living body according to [1], in which the mass ratio is 49/51 to 36/64.

[3] The composition for a living body according to [1] or [2], in which the mass ratio is 49/51 to 41/59.

[4] The composition for a living body according to any one of [1] to [3], in which the neutral acyl lipid further contains a neutral monoacyl lipid and a content of the neutral monoacyl lipid in the neutral acyl lipid is 40% to 90% by mass.

[5] The composition for a living body according to [4], in which the neutral monoacyl lipid contains glyceryl monooleate.

[6] The composition for a living body according to any one of [1] to [5], in which the phospholipid contains an ionic phospholipid.

[7] The composition for a living body according to any one of [1] to [6], in which the phospholipid contains phosphatidylcholine, and a content of the phosphatidylcholine in the phospholipid is at least 50% by mass.

[8] The composition for a living body according to any one of [1] to [7], in which a content of the alcohol having 4 or less carbon atoms or the polyalkylene oxide is 0.5 to 10 parts by mass with respect to 100 parts by mass of a total content of the neutral acyl lipid and the phospholipid.

[9] The composition for a living body according to any one of [1] to [8], in which a content of water is 0% by mass or more and 10% by mass or less with respect to a total amount of the composition for a living body.

The composition for a living body according to any one of [1] to [9], in which a reversed hexagonal columnar phase is formed by water absorption or moisture absorption.

The composition for a living body according to any one of [1] to [10], in which the composition for a living body is for a skin or a mucous membrane.

According to the present invention, it is possible to provide a composition for a living body, which has excellent spreadability on an object, has a high film formation rate in a case of being brought into contact with water to form a film, increases the moisture retention of an object adjacent to the formed film, and has excellent strength of the formed film.

### BRIEF DESCRIPTION OF THE DRAWINGS

FIG. 1 is photographs taken by observing, over time with a polarization microscope, the formation of a liquid crystal phase of the composition for a living body of Examples 1 to 4 and Comparative Examples 1 to 3 in the evaluation of the film formation rate of the present invention.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

Hereinafter, meaning of each description in the present specification will be explained.

In the present specification, the range indicated by using “to” means a range including both ends before and after “to”. For example, a range indicated by “A to B” includes A and B.

### [Composition for Living Body]

The composition for a living body according to the embodiment of the present invention is a composition for a living body, containing a neutral acyl lipid containing a neutral diacyl lipid, a phospholipid, and an alcohol having 4 or less carbon atoms or a polyalkylene oxide.

Each component contained in the composition for a living body according to the embodiment of the present invention will be described below.

### [Neutral Acyl Lipid]

The neutral acyl lipid means an electrically neutral acyl lipid. That is, the neutral acyl lipid does not include a cation moiety and an anion moiety. The acyl lipid means a lipid containing an acyl group.

### [Neutral Diacyl Lipid]

In the present invention, the neutral acyl lipid contains a neutral diacyl lipid. The neutral acyl lipid can further contain at least one or both of a neutral monoacyl lipid and a neutral triacyl lipid, in addition to the neutral diacyl lipid.

### [Content of Neutral Diacyl Lipid]

In the present invention, the content of the neutral diacyl lipid in the neutral acyl lipid is, with respect to the total amount of the neutral acyl lipid, more than 0% by mass (exceeding 0% by mass) and 50% by mass or less, and from the viewpoint that the effects of the present invention (particularly, the moisture retention, the spreadability, and the strength of the film) are more excellent, is preferably 10% to 50% by mass and more preferably 20% to 50% by mass.

### (Neutral Monoacyl Lipid)

In a case where the neutral acyl lipid further contains a neutral monoacyl lipid, from the viewpoint that the effects of the present invention (particularly, the moisture retention, the spreadability, and the strength of the film) are more excellent, the content of the neutral monoacyl lipid in the neutral acyl lipid is, with respect to the total amount of the neutral acyl lipid, preferably 40% to 90% by mass and more preferably 40% to 70% by mass.

The neutral monoacyl lipid preferably contains glyceryl monooleate.

### (Neutral Triacyl Lipid)

In the present invention, from the viewpoint that the effects of the present invention (particularly, the moisture retention, the spreadability, and the strength of the film) are more excellent, the content of the neutral triacyl lipid in the neutral acyl lipid is, with respect to the total amount of the neutral acyl lipid, preferably 0% to 10% by mass and more preferably 1% to 8% by mass.

The number of carbon atoms of the acyl group of the neutral acyl lipid is not particularly limited; however, the acyl group preferably has 6 to 32 carbon atoms and more preferably 16 to 22 carbon atoms.

The hydrocarbon group as the acyl group, excluding a carbonyl group, is preferably a saturated or unsaturated chain-type hydrocarbon group having 5 to 31 carbon atoms, and more preferably a saturated or unsaturated chain-type hydrocarbon group having 15 to 21 carbon atoms. Specific examples thereof include CH₃(CH₂)₁₄—, CH₃(CH₂)₇CH═CH(CH₂)₇—, and CH₃(CH₂)₄(CH═CHCH₂)₂(CH₂)₆—; however, the examples are not limited thereto.

In a case where a neutral acyl lipid has two or more acyl groups in one molecule, the kinds of acyl groups may be the same or different from each other.

Examples of the neutral acyl lipid include glycerol, diglycerol, sugar (for example, inositol), and a lipid obtained by forming an ester bond between a polyol such as succinic acid and a fatty acid. Among the above, an acyl glycerol is preferable and a glyceryl oleate is more preferable.

Examples of the acyl glycerol include monoacyl glycerol, diacyl glycerol, and triacyl glycerol.

In addition, examples of the glyceryl oleate include glyceryl monooleate, glyceryl dioleate, and glyceryl trioleate.

Here, the monoacyl glycerol and the glyceryl monooleate correspond to one aspect of the neutral monoacyl lipid described above, and the diacyl glycerol and the glyceryl dioleate correspond to one aspect of the neutral diacyl lipid described above, and the triacyl glycerol and the glyceryl trioleate correspond to one aspect of the neutral triacyl lipid.

In the present specification, the content proportions of the neutral monoacyl lipid, the neutral diacyl lipid, and the neutral triacyl lipid in the neutral acyl lipid are obtained from measurement with a high performance liquid chromatography (HPLC) method.

### HPLC Measurement Conditions

Column: Cadenza CD-C18 (4.6 mm×300 mm) (manufactured by Imtakt Corporation)
Eluent: water and tetrahydrofuran
Flow rate: 1.0 mL/min
Detection: Corona Charged Aerosol Detector (Corona CAD)
Column temperature: 50° C.

### [Phospholipid]

The composition for a living body according to the embodiment of the present invention contains a phospholipid.

The phospholipid is not particularly limited as long as it has a phosphate ester structure in the molecular structure thereof; however, a glycerophospholipid having glycerin as a skeleton and a sphingophospholipid having sphingosine as a skeleton are typical.

In both a case of the phospholipid being a glycerophosphoric acid and a case of the phospholipid being a sphingophospholipid, an acyl group derived from a fatty acid is contained in the molecule.

The number of carbon atoms of the acyl group of the phospholipid is not particularly limited; however, the acyl group preferably has 12 to 22 carbon atoms and more preferably 16 to 18 carbon atoms.

The hydrocarbon group as the acyl group, excluding a carbonyl group, is preferably a saturated or unsaturated chain-type hydrocarbon group having 11 to 21 carbon atoms, and more preferably a saturated or unsaturated chain-type hydrocarbon group having 15 to 17 carbon atoms. Specific examples of the hydrocarbon group include CH₃(CH₂)₁₄—, CH₃(CH₂)₇CH═CH(CH₂)₇—, and CH₃(CH₂)₄(CH═CHCH₂)₂(CH₂)₆—; however, the examples are not limited thereto.

In a case where a phospholipid has two or more acyl groups in one molecule, the kinds of acyl groups may be the same or different from each other.

Specific examples of the phospholipid include phosphatidylcholine. The acyl group of the phosphatidylcholine is preferably an acyl group derived from palmitic acid (CH₃(CH₂)₁₄COOH), oleic acid (CH₃(CH₂)₇CH═CH(CH₂)₇COOH), or linoleic acid (CH₃(CH₂)₄(CH═CHCH₂)₂(CH₂)₆COOH). Specific examples of the phosphatidylcholine include a PO phosphatidylcholine (a phosphatidylcholine having palmitic acid at the first position (the α position), oleic acid at the second position (the β position), and choline at the third position (the γ position)), a DL phosphatidylcholine (a phosphatidylcholine having linoleic acid at the first position (the α position), linoleic acid at the second position (the β position), and choline at the third position (the γ position)), and a dipalmitoyl phosphatidylcholine.

From the viewpoint that the water absorption rate of the composition for a living body according to the embodiment of the present invention can be improved or a columnar phase having a large domain size can be formed, the phospholipid preferably contains an ionic phospholipid. It is considered that the improvement of the water absorption rate of the composition for a living body and the like contributes to the further improvement of at least one of the film formation rate or the strength of the film. The above-described water absorption rate refers to a rate at which the composition for a living body according to the embodiment of the present invention absorbs water or moisture.

Examples of the ionic phospholipid include a phospholipid having a cationic moiety and an anionic moiety in one molecule, and specific examples thereof include phosphatidylcholine. Phosphatidylcholine has, as a cationic moiety, N⁺ derived from choline and P—O⁻ derived from phosphoric acid.

In a case where the phospholipid contains phosphatidylcholine, the content of the phosphatidylcholine in the phospholipid is preferably at least 50% by mass with respect to the total amount of the phospholipid. The upper limit of the content of phosphatidylcholine with respect to the total amount of phospholipids is not particularly limited, and examples thereof is 99% by mass.

### [Mass Ratio of Content of Neutral Acyl Lipid to Content of Phospholipid]

In the present invention, the mass ratio (neutral acyl lipid/phospholipid) of the content of the neutral acyl lipid to the content of the phospholipid is 54/46 to 30/70.

From the viewpoint that the effect of the present invention is more excellent, the above-described mass ratio is preferably 49/51 to 36/64 and more preferably 49/51 to 41/59.

### (Total Content of Neutral Acyl Lipid and Phospholipid)

The total content of the neutral acyl lipid and the phospholipid is preferably 77% to 99.5% by mass and more preferably 93% to 98% by mass with respect to the total amount of the composition for a living body according to the embodiment of the present invention.

The amount obtained by subtracting the above-described total content from the total amount of the composition for a living body according to the embodiment of the present invention may be used as the content of the alcohol having 4 or less carbon atoms or the polyalkylene oxide, or the total content of the alcohol having 4 or less carbon atoms and the polyalkylene oxide, which will be described later.

### [Alcohol Having 4 or Less Carbon Atoms or Polyalkylene Oxide]

The composition for a living body according to the embodiment of the present invention contains an alcohol having 4 or less carbon atoms or a polyalkylene oxide.

The composition for a living body according to the embodiment of the present invention has excellent moisture retention and excellent spreadability by containing an alcohol having 4 or less carbon atoms or a polyalkylene oxide.

The alcohol having 4 or less carbon atoms is not particularly limited as long as it is a compound having biocompatibility. The same applies to the polyalkylene oxide.

An alcohol having 4 or less carbon atoms can function as a solvent. The same applies to the polyalkylene oxide.

### [Alcohol Having 4 or Less Carbon Atoms]

The alcohol having 4 or less carbon atoms is a compound in which a hydroxy group is bonded to an aliphatic hydrocarbon group having 3 or less carbon atoms. The number of hydroxy groups included in one molecule of the alcohol having 4 or less carbon atoms is preferably 1 or 2.

Examples of the alcohol having 4 or less carbon atoms include monohydric alcohols such as ethanol; and dihydric alcohols such as propylene glycol and 1,3-butylene glycol.

### [Polyalkylene Oxide]

The polyalkylene oxide is a polymer having an oxyalkylene group as a repeating unit.

Terminals of the polyalkylene oxide may form a hydroxy group.

The polyalkylene oxide may have one or a plurality of hydroxy groups per molecule.

Examples of the polyalkylene oxide include polyoxyethylene polyol, polyoxypropylene polyol, and polyoxyethyleneoxypropylene polyol.

### (Content of Alcohol Having 4 or Less Carbon Atoms and the Like)

A content of the alcohol having 4 or less carbon atoms or the polyalkylene oxide is preferably 0.5 to 10 parts by mass and more preferably 2.0 to 8.0 parts by mass with respect to 100 parts by mass of the total content of the neutral acyl lipid and the phospholipid.

In a case where the composition for a living body according to the embodiment of the present invention contains the alcohol having 4 or less carbon atoms and the polyalkylene oxide, the total content of the alcohol having 4 or less carbon atoms and the polyalkylene oxide can be 1.0 to 20 parts by mass with respect to 100 parts by mass of the total content of the neutral acyl lipid and the phospholipid.

### (Other Components)

The composition for a living body according to the embodiment of the present invention can further contain a quaternary ammonium salt (excluding phosphatidylcholine) and water.

### (Quaternary Ammonium Salt)

The quaternary ammonium salt is an ionic compound consisting of a polyatomic ion having a positive charge, which is represented by a molecular formula NR₄⁻ (quaternary ammonium cation), and an anion. In NR₄⁺, R's each independently represent an alkyl group or an aryl group, and a plurality of R's may be the same as or different from each other. The anion is not particularly limited.

Examples of the quaternary ammonium salt include dioleoyloxytrimethylammonium propane chloride (DOTAP, N-[1-(2,3-dioleoyloxy) propyl]-N,N,N-trimethylammonium chloride), dioctadecenyltrimethylammonium propane chloride (DOTMA, N-[1-(2,3-dioleoyloxy) propyl)]-N,N,N-trimethylammonium chloride), didecyldimethylammonium chloride, didecyldimethylammonium bromide, dilauryldimethylammonium chloride, dicetyldimethylammonium chloride, dicetyldimethylammonium bromide, distearyldimethylammonium chloride, distearyldimethylammonium bromide, dioleyldimethylammonium chloride, dibehenyldimethylammonium chloride, dibehenyldimethylammonium bromide, dipalmitoylethyl hydroxyethylmonium metosulfate, and distearoylethyl hydroxyethylmonium metosulfate.

The composition for a living body according to the embodiment of the present invention may or may not contain water.

The content of the water is preferably 0% by mass or more and 10% by mass or less, and more preferably 0% by mass with respect to the total amount of the composition for a living body.

From the viewpoint of ensuring the transparency of the obtained film, it is preferable to exclude the aspect containing the carboxyvinyl polymer from the composition for a living body according to the embodiment of the present invention. That is, it is preferable that the composition for a living body according to the embodiment of the present invention does not contain the carboxyvinyl polymer. The carboxyvinyl polymer is a vinyl polymer having a carboxy group or a salt thereof. Examples of the carboxyvinyl polymer include a polymer having a repeating unit derived from acrylic acid and/or methacrylic acid as a main chain. The carboxyvinyl polymer may be crosslinked. For details of the carboxyvinyl polymer, WO2020/059543A can be referred to. The aspect containing the carboxyvinyl polymer may further include or may not include a amphiphilic block polymer, which will be described later.

In addition, it is preferable to exclude, from the composition for a living body of the present invention, an aspect containing an amphiphilic block polymer which consists of a hydrophilic segment and a hydrophobic segment and has a difference in C log P value between the hydrophilic segment and the hydrophobic segment of more than 1.00. That is, it is preferable that the composition for a living body according to the embodiment of the present invention does not contain the above-described amphiphilic block polymer. The amphiphilic block polymer is the same as “the amphiphilic block polymer which consists of a hydrophilic segment and a hydrophobic segment and has a difference in C log P value between the hydrophilic segment and the hydrophobic segment of more than 1.00” described in WO2020/202926A. The aspect including the above-described amphiphilic block polymer may further include or may not include the above-described carboxyvinyl polymer.

### (Method for Producing Composition for Living Body)

Examples of a manufacturing method of the composition for a living body according to the embodiment of the present invention include a method of mixing the neutral acyl lipid, the phospholipid, the alcohol having 4 or less carbon atoms or the polyalkylene oxide, and the other components which can be used as necessary. In the above-described mixing, the content of the neutral diacyl lipid may be more than 0% by mass and 50% by mass or less with respect to the total amount of the neutral acyl lipids, and a mass ratio of the content of the neutral acyl lipid to the content of the phospholipid may be 54/46 to 30/70.

The method of mixing is not particularly limited, and a conventionally known method can be used.

### (Liquid Crystal Phase)

The composition for a living body according to the embodiment of the present invention can form a liquid crystal phase by being brought into contact with water. In the present invention, as described above, the composition for a living body according to the embodiment of the present invention forms a liquid crystal phase, thereby the composition for a living body according to the embodiment of the present invention forms a film.

The composition for a living body according to the embodiment of the present invention can form quickly a liquid crystal phase in a short time even in a case of being brought in contact with a small amount of water, for example, water (moisture) in the atmosphere.

A thickness of the liquid crystal phase (film) formed of the composition for a living body according to the embodiment of the present invention is not particularly limited, but can be, for example, 0.1 μm to 1 mm.

In addition, the composition for a living body according to the embodiment of the present invention can be more strongly adhered to an object by being brought into contact with water.

### (Water)

Examples of the water to be brought into contact with the composition for a living body according to the embodiment of the present invention include water (moisture) in the atmosphere, water (moisture) in exhalation, pure water, and water contained in an aqueous fluid other than the water. Examples of the aqueous fluid other than water include saliva, tissue fluid, blood, and lymph.

In a case of using the composition for a living body according to the embodiment of the present invention, the amount of water to be brought into contact with the composition for a living body according to the embodiment of the present invention is not particularly limited, but is preferably 1,000% by mass or less and more preferably 500% by mass or less with respect to the total mass of the composition for a living body according to the embodiment of the present invention. The lower limit of the amount of the water with respect to the total mass of the composition for a living body according to the embodiment of the present invention, which is used in a case of being brought into contact with the composition for a living body according to the embodiment of the present invention, is not particularly limited, and examples thereof include more than 1% by mass.

A temperature at which the composition for a living body according to the embodiment of the present invention is brought into contact with water is not particularly limited, but is preferably 20° C. to 40° C. and more preferably 35° C. to 40° C.

The liquid crystal phase which can be formed by bringing the composition for a living body according to the embodiment of the present invention into the contact with water is not particularly limited, and is any one selected from the group consisting of a reversed hexagonal columnar (H2) phase, a hexagonal columnar (H1) phase, a lamellar (La) phase, a sponge (V2) phase, a bicontinuous cubic (L3) phase, and a mixed state of two or more thereof in many cases. The above-described liquid crystal phase preferably has a reversed hexagonal columnar phase (W/O hexagonal columnar phase) or a hexagonal columnar (H1) phase (O/W hexagonal columnar phase).

The composition for a living body according to the embodiment of the present invention preferably forms a reversed hexagonal columnar (H2) phase by water absorption or moisture absorption, and more preferably forms a reversed hexagonal columnar (H2) phase by moisture absorption.

The moisture absorption refers to the absorption of moisture. Examples of the moisture include water in the atmosphere and water in the exhalation.

In the present specification, the water absorption refers to the absorption of water (excluding moisture). Examples of the water (excluding moisture) include pure water and the aqueous fluid other than the water described above.

### (Application of Composition for Living Body)

The composition for a living body according to the embodiment of the present invention means a material which can be used for a living body. In a living body, the composition for a living body according to the embodiment of the present invention can be used, for example, for the purpose of assisting or repairing a part that does not exhibit an original function due to an injury, a disease, or the like (for example, an object such as a skin or a mucous membrane, the same applies to “portion” hereinafter), and a part where the function is decreased.

The composition for a living body according to the embodiment of the present invention can be preferably used for a skin or a mucous membrane (particularly, for an oral mucous membrane).

### (Method for Using Composition for Living Body)

Examples of the method for using the composition for a living body according to the embodiment of the present invention include a method in which the composition for a living body according to the embodiment of the present invention is disposed on a portion having the symptoms described above, and then the composition for a living body according to the embodiment of the present invention is brought into contact with water.

In a case where the composition for a living body according to the embodiment of the present invention is used on the skin, for example, the composition for a living body according to the embodiment of the present invention may be applied onto the skin in the atmosphere, and water or an aqueous solution containing water may be added to the composition for a living body according to the embodiment of the present invention as necessary.

The composition for a living body according to the embodiment of the present invention on the skin can form a liquid crystal phase by being brought into contact with, for example, any of water in the atmosphere, water sprayed by spraying, the above-described aqueous fluid, and exudate from the skin.

In a case where the composition for a living body according to the embodiment of the present invention is used for a mucous membrane, the composition for a living body according to the embodiment of the present invention may be disposed on the mucous membrane, and water or an aqueous solution containing water may be added to the composition for a living body according to the embodiment of the present invention as necessary.

The composition for a living body according to the embodiment of the present invention on the mucous membrane can form a liquid crystal phase by being brought into contact with, for example, any of water in the atmosphere, water in the exhalation, water sprayed by spraying, the above-described aqueous fluid, or water ingested.

In particular, in a case where the composition for a living body according to the embodiment of the present invention is applied to an oral mucous membrane, since the composition for a living body according to the embodiment of the present invention is adhered (applied) to the oral mucous membrane and thus brought into contact with any of water in the atmosphere, water in the exhalation, or water in the saliva to form the liquid crystal phase, the handling is simple. In addition, in a case where the amount of saliva is small, it is sufficient to supply the moisture by spraying water or artificial saliva after attaching the composition for a living body according to the embodiment of the present invention to the oral mucous membrane.

### (Phase Transition)

In the composition for a living body according to the embodiment of the present invention, the liquid crystal phase may undergo a phase transition by further absorbing water after absorbing moisture. Examples of the above-described phase transition include a phase transition from a reversed hexagonal columnar (H2) phase to a hexagonal columnar (H1) phase. Specifically, as described above, examples thereof include an aspect in which the moisture in a small amount of water (moisture) such as water in the atmosphere and water in exhalation is absorbed to form a liquid crystal phase (for example, a reversed hexagonal columnar (H2) phase), and then a larger amount of water than water (moisture) such as in the atmosphere from the air (moisture), such as water sprayed by spraying, artificial saliva, an aqueous fluid, exudate from a skin, and water ingested is absorbed to cause the above-described liquid crystal phase to undergo a phase transition to another liquid crystal phase (for example, a hexagonal columnar (H1) phase).

### EXAMPLES

Hereinafter, the present invention will be more specifically described according to Examples, but the present invention is not limited to Examples below.

### Examples 1 to 5 and Comparative Examples 1 to 6

### (Preparation of Composition for a Living Body)

Each composition for a living body was prepared by mixing the components shown in Table 1 in the amounts (parts by mass) shown in the same table.

In Comparative Example 6, Episil® (manufactured by Meiji Seika Pharma Co., Ltd.) was used as the composition for a living body. In the neutral acyl lipid contained in Episil®, the total amount of the neutral acyl lipids is a neutral diacyl lipid (glyceryl dioleate).

### <Evaluation>

The following evaluations were performed using each of the above-described compositions for a living body. The results are shown in Table 1.

### (Moisture Retention)

### Evaluation Method

An aqueous solution in which agar (Karikorikan (registered trademark), manufactured by Ina Food Industry Co., Ltd.) has been dissolved at 1% by mass concentration by heating at 90° C. for 10 minutes was put into a glass dish in which the weight was measured in advance (weight of X g, in unit of g), and allowed to stand until the solution reached room temperature, thereby forming an agar gel having a concentration of 1% by mass.

Each composition for a living body (thickness of 200 μm) was placed to cover the entire surface of the above-described agar gel, and the weight (Yg) of each composition for a living body was measured.

A weight (Z₀ g) of the glass dish containing the agar gel and the composition for a living body was measured, the glass dish was allowed to stand in a constant-temperature tank set at 25° C. and 50% rh (relative humidity) for 24 hours, and a weight (Z g) of the entire glass dish was measured after 24 hours.

The water retention rate (%) of the agar gel was calculated by Expression ((Z−X−Y)/(Z₀−X−Y)×100).

### Evaluation Criteria

In a case where the water retention rate of the agar gel was 90% or more, it was evaluated that the moisture retention (property of suppressing the evaporation of water in the object) of the object adjacent to the formed film was extremely excellent, and this was indicated as “A”.

In a case where the water retention rate was 50% or more and less than 90%, it was evaluated that the above-described moisture retention was slightly excellent, and this was indicated as “B”.

On the other hand, in a case where the water retention rate was less than 50%, it was evaluated that the above-described moisture retention was low, and this was indicated as “C”.

### (Spreadability)

### Evaluation Method

A rheometer device (MCR302) and a measurement jig CP25 were used. Each of the above-described compositions for a living body was set on a base of a rheometer device, and the viscosity of each composition for a living body was measured under the conditions of a measurement temperature of 25° C., 50% rh (relative humidity), and a shear rate (1/s) of 0.1 to 1,000.

### Evaluation Criteria

In a case where the viscosity at a shear rate of 0.1 (1/s) was less than 5,000 cPs, it was evaluated that the spreadability (ease of spreading) on the object was extremely excellent, and this was indicated as “A”.

In a case where the above-described viscosity was 5,000 cPs or more and less than 1,000,000 cPs, it was evaluated that the spreadability were slightly excellent, and this was indicated as “B”.

On the other hand, in a case where the viscosity was 1,000,000 cPs or more, it was evaluated that the spreadability was low, and this was indicated as “C”.

### (Film Formation Rate)

### Evaluation Method

Each composition for a living body was applied onto a slide glass to have a thickness of 200 μm under conditions of 25° C. and 50% rh.

After the application, the composition for a living body on the slide glass was observed with a polarizing microscope after 1 minute, 30 minutes, 1 hour, and 24 hours.

In the above observation, a time required for the composition for a living body on the slide glass to form a liquid crystal phase (time required for the field of view of the polarizing microscope to change from a dark field to a bright field) was recorded. In a case where the composition for a living body forms a liquid crystal phase, a field of view of a polarizing microscope is a bright field.

### Evaluation Criteria

In a case where the time until the composition for a living body formed a liquid crystal phase was within 1 minute from the application, it was evaluated that the speed of the film formation was very high, and this was indicated as “A”.

Although the above-described A did not apply, in a case where the liquid crystal phase was formed within 30 minutes from the application, it was evaluated that the speed of the film formation was slightly fast, and this was indicated as “B”.

On the other hand, in a case where the liquid crystal phase was formed within 1 hour from the application, which did not correspond to A and B described above, it was evaluated that the rate of the film formation was slightly slow, and this was indicated as “C”.

In a case where the liquid crystal phase was not formed at a point of time 1 hour after the application, which did not correspond to any of A to C described above, it was evaluated that the rate of the film formation was extremely slow, and this was indicated as “D”.

In the evaluation of the present example, in all Examples and Comparative Examples, the liquid crystal phase was formed 24 hours after the application.

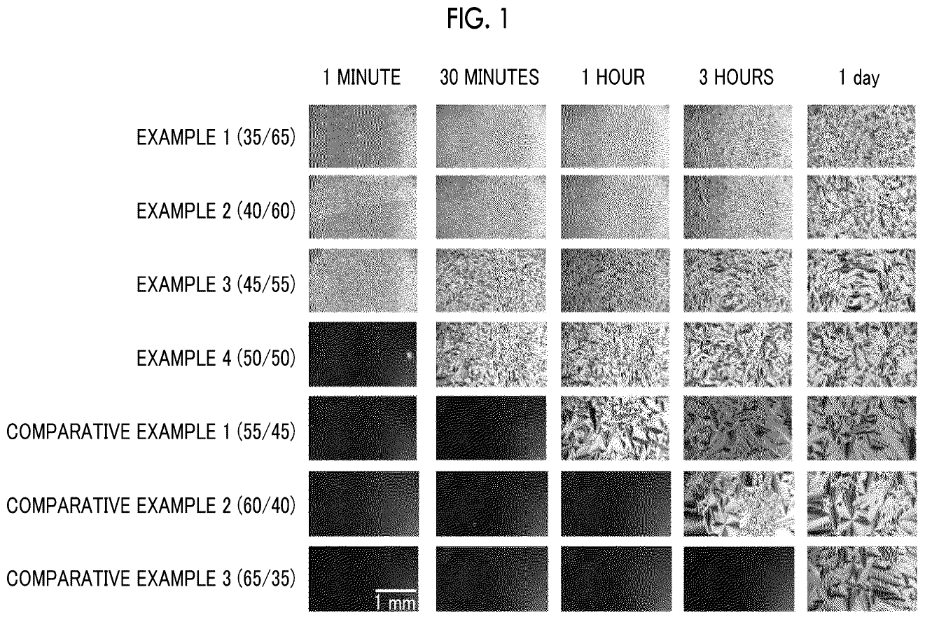

### FIG. 1

FIG. 1 is photographs taken by observing, over time with a polarization microscope (Nikon ECLIPSE E600 POL), the formation of a liquid crystal phase of the composition for a living body of Examples 1 to 4 and Comparative Examples 1 to 3 in the evaluation of the film formation rate of the present invention (magnification of 50 times).

In FIG. 1, for each of Examples and Comparative Examples, the photographs taken 1 minute, 30 minutes, 1 hour, 3 hours, and 24 hours (1 day) after the application of the composition for a living body were arranged in order.

“(x/x)” shown after each example is a mass ratio (neutral acyl lipid/phospholipid) of the content of the neutral acyl lipid to the content of the phospholipid in each example. For example, “Example 1 (35/65)” indicates that the above-described mass ratio (neutral acyl lipid/phospholipid) is 35/65 in Example 1.

From the results shown in FIG. 1 as a whole, a correlation relationship was confirmed in which the film formation rate was higher as the proportion of the phospholipid in the mass ratio (neutral acyl lipid/phospholipid) was higher.

### (Strength of Film)

### Evaluation Method

A rheometer device (MCR302) and a measurement jig PP25 were used. Each composition for a living body was applied to the base in advance in a size of a diameter of 25 mm and a thickness of 200 μm, and the base was set in a rheometer device. Water was sprayed 3 times (the amount of water sprayed 3 times was 0.3 mL) onto the composition for a living body, which was applied as described above, using a trusco finger spray (TFSB-20), and the composition was allowed to stand for 1 minute.

After 1 minute, the strain dispersion was measured from 0.001% to 1,000% at a measurement temperature of 25° C., a measurement GAP of 200 μm, a frequency of 1 Hz, and N_{f}=1 N, and a value of the storage elastic modulus G′ at a shear strain (0.1%) was obtained.

### Evaluation Criteria

In a case where the storage elastic modulus G′ was 15,000 Pa or more, it was evaluated that the strength of the film formed from the composition for a living body was extremely excellent, and this was indicated as “A”.

In a case where the storage elastic modulus G′ was 5,000 Pa or more and less than 15,000 Pa, it was evaluated that the strength of the film was slightly excellent, and this was indicated as “B”.

On the other hand, in a case where the storage elastic modulus G′ was less than 5,000 Pa, it was evaluated that the strength of the film was low, and this was indicated as “C”.

### (Confirmation of Liquid Crystal Phase)

### Evaluation Method

Each of the prepared compositions for a living body was applied onto a slide glass to have a thickness of 200 μm, and small-angle X-ray scattering (SAXS) measurement was performed using a sample in which the above-described slide glass had been allowed to stand at 25° C. and 50% rh (relative humidity) for 24 hours or more, and the liquid crystal structure was determined. For the SAXS measurement, a small-angle X-ray scattering measuring device Nanopix (Cu Ka, 40 kV/30 mA) manufactured by Rigaku Corporation was used. The results of the SAXS measurement are shown by a scattering curve using a scattering intensity as the ordinate and a scattering vector length (q/nm-1) as the abscissa. For each peak on the obtained scattering curve, a ratio of the scattering vector length (q/nm-1) was measured to identify the liquid crystal structure.

In a case where at least three peaks were observed on the scattering curve and the ratio of the scattering vector lengths of the peaks was 1:√3:√4, it was determined that the liquid crystal phase was a reversed hexagonal columnar phase. The ratio of the scattering vector lengths of the three peaks of 1:√3:√4 is specific to the reversed hexagonal columnar phase.

### Evaluation Results

In a case where the liquid crystal phase was a reversed hexagonal columnar phase, this was indicated as “H2”.

In a case where the liquid crystal phase was not the reversed hexagonal columnar phase, this was indicated as “not H2”.

As described above, in the evaluation of the present example, in all Examples and Comparative Examples, the liquid crystal phase was formed 24 hours after the application.

TABLE 1

| | Example 1 | Example 2 | Example 3 | Example 4 | Example 5 |
|---|---|---|---|---|---|
| Neutral acyl lipid 1 | 35 | 40 | 45 | 50 | |
| (content of neutral diacyl lipid | | | | | |
| in neutral acyl lipid 1 is 50% | | | | | |
| by mass or less, mono:di:tri = | | | | | |
| 49:45:6) | | | | | |
| Neutral acyl lipid 2 | | | | | 45 |
| (content of neutral diacyl lipid | | | | | |
| in neutral acyl lipid 2 is 50% | | | | | |
| by mass or less, mono:di:tri = | | | | | |
| 95:5:0) | | | | | |
| (Comparative) Neutral acyl | | | | | |
| lipid 3 | | | | | |
| (content of neutral diacyl lipid | | | | | |
| in neutral acyl lipid 3 is more | | | | | |
| than 50% by mass) | | | | | |
| Phospholipid | 65 | 60 | 55 | 50 | 55 |
| Propylene glycol | 5 | 5 | 5 | 5 | 5 |
| (Comparative) Oleyl alcohol | | | | | |
| Moisture retention | B (69%) | B (88%) | A (92%) | A (92%) | B (63%) |
| Spreadability | B (20625) | B (10509) | A (3172) | A (1869) | B (8.67 × 10⁵) |
| (unit: cPs) | | | | | |
| Film formation rate | A (≤1 min) | A (≤1 min) | A (≤1 min) | B (≤30 min) | A (≤1 min) |
| Strength of film | B (13362) | A (18227) | A (17369) | B (6197) | B (8004) |
| (unit: Pa) | | | | | |
| Confirmation of liquid crystal | H2 | H2 | H2 | H2 | not H2 |
| phase | | | | | |

| | Comparative | Comparative | Comparative | Comparative | Comparative | Comparative |
|---|---|---|---|---|---|---|
| Subsequent to Table 1 | Example 1 | Example 2 | Example 3 | Example 4 | Example 5 | Example 6 |
| Neutral acyl lipid 1 | 55 | 60 | 65 | | 45 | Episil^{(R)} |
| (content of neutral diacyl lipid | | | | | | (content of neutral |
| in neutral acyl lipid 1 is 50% | | | | | | diacyl lipid in |
| by mass or less, mono:di:tri = | | | | | | neutral acyl lipid is |
| 49:45:6) | | | | | | more than 50% by |
| Neutral acyl lipid 2 | | | | | | mass) |
| (content of neutral diacyl lipid | | | | | | |
| in neutral acyl lipid 2 is 50% | | | | | | |
| by mass or less, mono:di:tri = | | | | | | |
| 95:5:0) | | | | | | |
| (Comparative) Neutral acyl | | | | 45 | | |
| lipid 3 | | | | | | |
| (content of neutral diacyl lipid | | | | | | |
| in neutral acyl lipid 3 is more | | | | | | |
| than 50% by mass) | | | | | | |
| Phospholipid | 45 | 40 | 35 | 55 | 55 | |
| Propylene glycol | 5 | 5 | 5 | 5 | | |
| (Comparative) Oleyl alcohol | | | | | 5 | |
| Moisture retention | A (95%) | A (93%) | A (97%) | A (96%) | C (44%) | A (90%) |
| Spreadability | A (1212) | A (711) | A (425) | B (9211) | C (1.04 × 10⁷) | A (94) |
| (unit: cPs) | | | | | | |
| Film formation rate | C (≤1 hour) | D (>1 hour) | D (>1 hour) | A (≤1 min) | A (≤1 min) | C |
| Strength of film | C (59) | C (0) | C (0) | C (1382) | A (30097) | C (17) |
| (unit: Pa) | | | | | | |
| Confirmation of liquid crystal | H2 | H2 | H2 | H2 | H2 | not H2 |
| phase | | | | | | |

Details of each component in Table 1 are as follows.

### (Neutral Acyl Lipid)

Neutral acyl lipid 1: a neutral acyl lipid containing a neutral monoacyl lipid, a neutral diacyl lipid, and a neutral triacyl lipid in a mass ratio of 49:45:6, in which the neutral monoacyl lipid contains glyceryl monooleate. Manufactured by Tokyo Chemical Industry Co., Ltd.
Neutral acyl lipid 2: a neutral acyl lipid having a mass ratio between a neutral monoacyl lipid, a neutral diacyl lipid, and a neutral triacyl lipid of 95:5:0, in which the neutral monoacyl lipid contains glyceryl monooleate. GLYMOIST-MO (manufactured by NOF CORPORATION)
(Comparative) Neutral acyl lipid 3: neutral acyl lipid containing neutral monoacyl lipid, neutral diacyl lipid, and neutral triacyl lipid in a mass ratio of 3:78:19. Manufactured by FUJIFILM Wako Pure Chemical Corporation

### (Phospholipid)

Phospholipid: phosphatidylcholine. Lipoid P100 (manufactured by Lipoid GmbH). The content of the phosphatidylcholine was 97.3% by mass in the above product.

### (Solvent)

Propylene glycol (manufactured by Tokyo Chemical Industry Co., Ltd.)
(Comparative) Oleyl alcohol (manufactured by Tokyo Chemical Industry Co., Ltd.)

### Comparative Example 6

Episil®: manufactured by Meiji Seika Pharma Co., Ltd. In the neutral acyl lipid contained in Episil®, the total amount of the neutral acyl lipids is a neutral diacyl lipid (glyceryl dioleate). Since the content of the neutral diacyl lipid in the neutral acyl lipid is more than 50% by mass, Epishell® does not correspond to the composition for a living body according to the embodiment of the present invention.

From the results shown in Table 1, in Comparative Examples 1 to 3 in which the mass ratio (neutral acyl lipid/phospholipid) of the content of the neutral acyl lipid to the content of the phospholipid was out of 54/46 to 30/70, the rate of the film formation was slow and the strength of the obtained film was low.

In Comparative Examples 4 and 6 in which the content of the neutral diacyl lipid in the neutral acyl lipid exceeded 50% by mass in the neutral acyl lipid, the strength of the obtained film was low. In Comparative Example 6, furthermore, the rate of the film formation was slow.

In Comparative Example 5, in which neither the predetermined alcohol nor the polyalkylene oxide was contained and instead oleyl alcohol was contained, the spreadability on the object was low and the moisture retention of the object was not retained.

On the other hand, the composition for a living body according to the embodiment of the present invention has excellent spreadability on an object, has a high film formation rate in a case of being brought into contact with water to form a film, increases the moisture retention of an object adjacent to the formed film, and has excellent strength of the formed film.

Among these, from the comparison of Examples 1 to 4, it was confirmed that in a case where the mass ratio of the content of the neutral acyl lipid to the content of the phospholipid was 49/51 to 36/64 (more preferably, 49/51 to 41/59), the effect was more excellent.

In addition, from the comparison between Examples 3 and 5, it was confirmed that, in a case where the content of the neutral monoacyl lipid in the neutral acyl lipid was 40% to 90% by mass, the effect was more excellent.

What is claimed is:

1. A composition for a living body, comprising:
a neutral acyl lipid containing glyceryl oleate;
a phospholipid; and
an alcohol having 4 or less carbon atoms or polyalkylene oxide,
wherein a content of a neutral diacyl lipid in the neutral acyl lipid is more than 0% by mass and 50% by mass or less,
a mass ratio of a content of the neutral acyl lipid to a content of the phospholipid is 54/46 to 30/70, and
the composition for a living body is for a skin or a mucous membrane.

2. The composition for a living body according to claim 1,
wherein the mass ratio is 49/51 to 36/64.

3. The composition for a living body according to claim 1,
wherein the mass ratio is 49/51 to 41/59.

4. The composition for a living body according to claim 1,
wherein the neutral acyl lipid further contains a neutral monoacyl lipid, and
a content of the neutral monoacyl lipid in the neutral acyl lipid is 40% to 90% by mass.

5. The composition for a living body according to claim 4,
wherein the neutral monoacyl lipid contains glyceryl monooleate.

6. The composition for a living body according to claim 1,
wherein the phospholipid contains an ionic phospholipid.

7. The composition for a living body according to claim 1,
wherein the phospholipid contains phosphatidylcholine, and
a content of the phosphatidylcholine in the phospholipid is at least 50% by mass.

8. The composition for a living body according to claim 1,
wherein a content of the alcohol having 4 or less carbon atoms or the polyalkylene oxide is 0.5 to 10 parts by mass with respect to 100 parts by mass of a total content of the neutral acyl lipid and the phospholipid.

9. The composition for a living body according to claim 1,
wherein a content of water is 0% by mass or more and 10% by mass or less with respect to a total amount of the composition for a living body.

10. The composition for a living body according to claim 1,
wherein a reversed hexagonal columnar phase is formed by water absorption or moisture absorption.

11. The composition for a living body according to claim 2,
wherein the mass ratio is 49/51 to 41/59.

12. The composition for a living body according to claim 2,
wherein the neutral acyl lipid further contains a neutral monoacyl lipid, and
a content of the neutral monoacyl lipid in the neutral acyl lipid is 40% to 90% by mass.

13. The composition for a living body according to claim 12,
wherein the neutral monoacyl lipid contains glyceryl monooleate.

14. The composition for a living body according to claim 2,
wherein the phospholipid contains an ionic phospholipid.

15. The composition for a living body according to claim 2,
wherein the phospholipid contains phosphatidylcholine, and
a content of the phosphatidylcholine in the phospholipid is at least 50% by mass.

16. The composition for a living body according to claim 2,
wherein a content of the alcohol having 4 or less carbon atoms or the polyalkylene oxide is 0.5 to 10 parts by mass with respect to 100 parts by mass of a total content of the neutral acyl lipid and the phospholipid.

17. The composition for a living body according to claim 2,
wherein a content of water is 0% by mass or more and 10% by mass or less with respect to a total amount of the composition for a living body.

18. The composition for a living body according to claim 2,
wherein a reversed hexagonal columnar phase is formed by water absorption or moisture absorption.

19. The composition for a living body according to claim 3,
wherein the neutral acyl lipid further contains a neutral monoacyl lipid, and
a content of the neutral monoacyl lipid in the neutral acyl lipid is 40% to 90% by mass.

20. The composition for a living body according to claim 19,
wherein the neutral monoacyl lipid contains glyceryl monooleate.
